(19) United States
(12) Patent Application Publication
(10) Pub. No.: US 2009/0004252 A1
(43) Pub. Date: Jan. 1, 2009

(54) Skin Coating Composition and Uses Thereof

(76) Inventors: Linda Lowndes, Queensland (AU); Leslie Pascoe, New South Wales (AU)

Correspondence Address: CANTOR COLBURN, LLP, 20 Church Street, 22nd Floor, Hartford, CT, 06103, US

(21) Appl. No.: 11/658,941

(22) PCT Filed: Jul. 29, 2005
(86) PCT No.: PCT/AU2005/001120

§ 371 Date: Sep. 25, 2007

(30) Foreign Application Priority Data
Jul. 29, 2004 (AU) 2004904224
Jul. 29, 2004 (AU) 2004904225

(51) Int. Cl. A61K 9/70; A61K 8/02; A61P 17/00; A61Q 17/04; A61Q 19/00

(52) U.S. Cl. 424/443; 424/63; 424/59

(57) ABSTRACT

The present invention relates to a composition for coating the skin wherein the composition forms a water-proof flexible film on the skin. Preferably, the flexible film is durable and is capable of remaining adhered to the skin for 1 to 3 days. The composition may include (i) one or more pigments and/or dyes, or (ii) one or more sunscreen agents or (iii) one or more therapeutic agents for preventing or treating disorders of the skin. The coating composition comprises resin and flexibilizer (platicizer). Any suitable type of natural or synthetic resin may be used, preferably the resin is shellac or other natural gum derivative. A suitable flexiblizer is an animal, vegetable, mineral or synthetic oil, preferably castor oil, pine oil, rosehip oil or soya bean oil or mixture thereof. The composition optionally includes a diluent such as ethanol, a cellulosic preparation such as ethyl cellulose, an anti-agglomerating or dispersing agent and a thickener. The composition may be in the form of a paste, cream, gel, liquid or aerosol and may be used as a bandage or artificial skin.

### FIELD OF THE INVENTION

The present invention generally relates to a composition for coating skin wherein the composition forms a durable waterproof flexible film on skin. The composition may include different ingredients for different applications. In one embodiment, the composition includes pigment and/or dye and may be used to coat a skin blemish such that the skin blemish blends in with the surrounding skin. In another embodiment, the composition includes a sunscreen agent and may be used as a sunscreen. In yet another embodiment, the composition includes a therapeutic agent for treating the skin.

The subject matter of this application is related to the applicant's co-pending International Patent Application filed on 29 Jul. 2005 and entitled “A Colour Compensating System” as well as Australian Provisional Patent Application No. 2004904225, the entire contents of which are herein incorporated by cross-reference.

### BACKGROUND OF THE INVENTION

Compositions for coating skin are known. Some are in the form of cosmetics for masking skin discoloration and skin blemishes, others are in the form of sunscreens for providing protection against the adverse effects of solar radiation, and yet others are in the form of skin care products for promoting healthy skin.

Some of the known skin coating compositions have the disadvantage that they are not long-lasting on the skin and may require frequent reapplication. Some compositions may be readily rubbed off the skin, whereas other compositions may be readily washed off the skin by way of sweat or when in contact with water.

### SUMMARY OF THE INVENTION

The present invention broadly relates to a coating composition that, when applied to the skin, forms a durable waterproof (i.e. impervious to water) flexible film.

According to a first aspect of the present invention, there is provided a coating composition for skin, said composition comprising resin and flexibilizer, wherein upon application to skin, said composition forms a waterproof flexible film.

According to a second aspect of the invention, there is provided a method for coating skin comprising the steps of:

(i) coating skin with a composition comprising resin and flexibilizer; and

(ii) allowing the composition to form a waterproof flexible film over the skin.

Preferably, the flexible film is durable and is capable of remaining adhered to the skin for 1 to 3 days and more preferably up to about 5 days. The durability of the film will depend, inter alia, on the composition of the film, the properties of the skin and the environmental conditions to which the skin and film are subjected.

The properties of the film will depend on the choice and quantity of resin and flexibilizer. The properties of the film may be adjusted as necessary by changing the ingredients of the composition as well as by varying the relative amounts of ingredients. Other ingredients (eg. diluent, thickener and adhesive) may be added to the composition, and these will also affect the properties of the film.

The composition may be, for example, in the form of a paste, cream, gel, liquid or aerosol. The film may be opaque, translucent or transparent. The composition may be used as a bandage or as artificial skin.

The composition may include a diluent. Any suitable type of diluent may be used. The composition may include more than one type of diluent. Any suitable quantity of diluent may be used. For instance, the composition may contain little diluent if applied to the skin as a paste, e.g. using a brush or sponge. The composition may contain more diluent if applied to the skin as a liquid, e.g. using an airbrushing gun. The composition may be in the form of an aerosol, packaged under pressure with a suitable gaseous propellant. The diluent may be evaporative. Preferably, the diluent is an alcohol, such as ethanol, which will readily evaporate. The alcohol may be denatured or non-denatured. Preferably, ethanol (denatured with IPA) is present in the composition in an amount of about 20-80% weight by weight (w/w).

Any suitable type of resin may be used. The composition may include more than one type of resin. The resin may be of natural or synthetic origin. Any suitable quantity of resin may be used. The resin may be a natural alcohol-soluble resin such as shellac or other natural gum derivative. Preferably, the resin is de-waxed (blonde) bleached shellac. More preferably, shellac is present in the composition in an amount of about 5-30% w/w.

Any suitable type of flexibilizer (plasticiser) may be used to impart flexibility to the film and to hinder flaking of the film from the skin. The composition may include more than one type of flexibilizer. Any suitable quantity of flexibilizer may be used. A suitable flexibilizer may be oil. The oil may be of animal, vegetable, mineral or synthetic origin. The oil may be, for example, castor oil, pine oil, eucalyptus oil, ti-tree oil, rosehip oil or soya bean oil, or a mixture thereof. Preferably, oil is present in the composition in an amount of about 1-70% w/w.

The composition may include a cellulosic preparation. A cellulosic preparation is an example of another suitable flexibilizer. Any suitable type of cellulosic preparation may be used. The cellulosic preparation may comprise, for example, ethyl cellulose or sucrose acetate isobutyrate. Preferably, a cellulosic preparation is present in the composition in an amount of about 2-20% w/w.

The composition may include an adhesive for improving attachment of the film to the skin. Any suitable type of adhesive may be used. The composition may include more than one type of adhesive. Any suitable quantity of adhesive may be used. Preferably, the adhesive is a cellulosic preparation such as, for example, ethyl cellulose or sucrose acetate isobutyrate.

The composition may include an anti-agglomeration agent or dispersing agent. Any suitable agent or agents may be used. A suitable agent may be a blend of neutralised acid esters of phosphoric acid together with 2-(2-butoxyethoxy)ethanol.

The composition may include thickener to increase the viscosity of the composition. Any suitable type of thickener may be used. The composition may include more than one type of thickener. Any suitable quantity of thickener may be used. A suitable thickener may include, for example, one or more of the following: a cellulosic preparation or an inorganic thickener such as silicon dioxide (fumed silica), castor oil derivatives, quaternium ammonium compound of bentonite, zinc stearate, nano zinc oxide (Zinclear™), inorganic thixotrope or modified clays. Preferably, inorganic thickener is present in the composition in an amount of about 0.5-10% w/w.

The composition may include a preservative for extending the shelf life of the composition. Any suitable type of preservative may be used. The composition may include more than one type of preservative. Any suitable quantity of preservative may be used. A suitable preservative may include, for example, one or more of the following: butylated hydroxytoluene (BHT), butylated hydroxyanisole, hydroquinone and methylhydroquinone. Preferably, preservative is present in the composition in an amount of about 0.5-5% w/w.

The film is preferably highly resistant to being washed off with water. The film may remain intact even when immersed in hot water. The film preferably bonds to the skin when immersed in saltwater. The film is preferably highly resistant to being rubbed off. The film may remain attached to the skin even if a shaver is scraped over the film. If more than one film layer is applied to the skin, the film layers may bond to one another. Preferably, the film enables the skin to breathe. It appears that the skin is able to breathe in that the film does not completely seal sweat pores of the skin as well as perhaps hair follicles of the skin.

According to a preferred form of the invention, the coating composition comprises:

about 5-30% w/w natural gum derivative;

about 50-80% w/w alcohol;

about 2-70% w/w oil;

about 2-10% w/w cellulosic preparation; and

about 0.5-10% w/w inorganic thickener.

Preferably, the natural gum derivative is shellac, the alcohol is denatured ethanol, the oil is castor oil (7-65% w/w) or a mixture of castor oil (7-65% w/w) and rosehip oil (0.1-5% w/w), the cellulosic preparation is ethyl cellulose, and the inorganic thickener is silicon dioxide.

Preferably, the preferred form further comprises about 0.5-3% w/w preservative, such as BHT.

The coating composition according to the first aspect of the invention may include different ingredients for different applications. For example, the coating composition may include at least one pigment and/or dye and be used to coat discoloured skin or a skin blemish such that the discoloured skin or skin blemish blends with the surrounding skin. The skin discoloration/skin blemish may be due to, for example, a birthmark, a mole, a basal cell carcinoma, vitiligo, a scar, a burn, pigmentation, acne, a vein, tattoo, eczema, dermatitis or bruising. Alternatively, such a composition may be used to simply change the skin colour of an individual.

Alternatively or additionally, the coating composition may include at least one sunscreen agent and be used as a sunscreen. The sunscreen may protect individuals against premature ageing of skin, skin cancer and other harmful effects of solar radiation.

Alternatively or additionally, the coating composition may include at least one therapeutic agent for preventing or treating disorders of the skin—for example, cuts, inflammation or infections.

According to a third aspect of the present invention, there is provided a coloured coating composition for skin, said composition comprising resin, flexibilizer and pigment and/or dye, wherein upon application to skin, said composition forms a waterproof flexible coloured film.

According to a preferred form of the invention, the coloured coating composition comprises:

about 5-30% w/w natural gum derivative;

about 50-80% w/w alcohol;

about 2-70% w/w oil;

about 2-10% w/w cellulosic preparation;

about 0.5-10% w/w inorganic thickener; and

at least one pigment and/or dye.

Any suitable type of pigment or dye may be used. The composition may include more than one type of pigment and/or dye, depending on the desired colour for the film. Any suitable quantity of pigment or dye may be used. Preferably, the pigment and/or dye is present in amount of about 0.01-10% w/w. A suitable pigment or dye may include, for example, one or more of the following:
Natural or synthetic iron oxides, black, red, yellow, brown, blended in various ratios.
Rutile titanium dioxide (micronised).
Zinc oxide (micronised).
Ultramarine blue (micronised).
Mixed metal oxide (cobalt) blue, black, turquoise, green.
Bon arymadide red pigments.
Bon red (calcium).
Rubine toners.
Arylamide yellows.
Transparent iron oxide pigments.
Phthalocyanine blues.
Dioxazine violets.
Pearlescent pigments in various colours, including white, copper, bronze.
Solvent dyes red, orange, yellow, blue, violet, brown, black.

Preferably, the pigment is micronised and uniformly dispersed throughout the film.

According to a fourth aspect of the present invention, there is provided a method for coating a skin discoloration or skin blemish such that the skin discoloration or blemish blends with the surrounding skin, said method comprising the steps of:

(i) coating a skin discoloration or blemish with a composition comprising resin, flexibilizer, and pigment and/or dye; and

(ii) allowing the composition to form a waterproof flexible coloured film over the skin discoloration or blemish.

The method may further comprise the step of (iii) applying a fixing powder to the coated skin discoloration or blemish to create a matte effect and to produce a more natural skin appearance. The fixing powder may be applied immediately after application of the composition and then reapplied after washing the skin. The application of a fixing powder is preferred if the composition is applied to the skin as a spray.

The fixing powder may be of any suitable composition. The fixing powder can comprise talc, kaolin, zinc stearate, silicone oil (dimethicone), propylene glycol, and one or more preservatives such as, for instance, methylparaben, propylparaben, ethylparaben, butylparaben and mydazolidinyl urea. Talc and kaolin are opaquing agents. Zinc stearate is a lubricant. Dimethicone aids in spreading and rubbing of the powder on the skin and acts as a barrier preventing water penetration. Propylene glycol is a solvent. The fixing powder may also reduce any stickiness of an exposed surface of the film.

In a preferred form the fixing powder comprises:

about 93% w/w talc;

about 4.7% w/w zinc stearate;

about 1.4% w/w silicone oil; and

about 0.9% w/w Unigerm G2™ (mixture of methylparaben, ethylparaben, propylene glycol and mydazolidinyl urea).

The method preferably comprises the step of cleaning the skin (of natural oils, cosmetics etc.) before applying the coating composition.

The method may comprise additional initial steps of:

measuring at least one colour property of the skin discoloration or blemish;

measuring at least one colour property of the surrounding skin; and

using the measured at least one colour property of the skin discoloration or blemish and the at least one colour property of the surrounding skin to formulate a composition having a compensatory colour such that the skin discoloration or blemish when coated with the composition blends with the surrounding skin.

Details of these additional initial steps may be found in the applicant's co-pending International Patent Application entitled “A Colour Compensating System” as well as in Provisional Patent Application No. 2004904225.

The coloured coating composition preferably has a degree of translucency when applied to the skin discoloration or blemish and is not of identical colour to the surrounding skin. The present inventor has found that previous attempts to cover discoloured skin/blemishes by selecting an opaque cosmetic coating that is exactly the same colour as the surrounding skin and subsequently applying that coating to the skin discoloration/blemish to hide or mask the discoloration/blemish resulted in an unnatural and overly-made up, almost plasticky, look. Rather than simply trying to hide the discoloration/blemish, the translucent coating can have an additive or complementary effect with the underlying discoloration/blemish such that the discoloration/blemish, when coated, has a more natural appearance whilst still matching the appearance of the surrounding skin.

According to a fifth aspect of the present invention, there is provided a sunscreen composition for skin, said composition comprising resin, flexibilizer and sunscreen agent, wherein upon application to skin, said composition forms a waterproof flexible film having a defined sun protection factor (SPF) value.

Any suitable type of sunscreen agent may be used. The composition may include one or more sunscreen agents. Suitable sunscreen agents include titanium dioxide and zinc oxide. Preferably, the composition includes zinc oxide. If a clear sunscreen is desired, the zinc oxide may have a particle size of about 5-100 nm, and more preferably a particle size of about 20-40 nm. If a white opaque sunscreen is desired, the zinc oxide may have a larger particle size.

The composition may have any defined SPF value, depending on its intended use. Preferably, the SPF value is at least 15 and more preferably at least 30. The composition may be applied to the skin in layers so as to exceed an SPF value of 30. A multilayer film may provide an SPF value of over 1000.

Preferably, the sunscreen film is durable in that it need not be reapplied for at least 1 to 3 days and up to about five days.

The sunscreen composition may further include a pigment and/or dye for coating discoloured skin or skin blemishes, or for producing an opaque film. Individuals suffering from xeroderma pigmentosum may benefit from a long-lasting opaque sunscreen having a high SPF value. Such individuals may benefit from a multilayer film, whether the films are of the same composition or of different compositions.

According to a preferred form of the invention, the sunscreen composition comprises:

about 5-30% w/w natural gum derivative;

about 20-60% w/w alcohol;

about 10-60% w/w sunscreen agent;

about 2-20% w/w oil; and

about 2-15% w/w cellulosic preparation.

Preferably, the natural gum derivative is shellac, the alcohol is denatured ethanol, the oil is castor oil (2-20% w/w) or a mixture of castor oil (2-15% w/w) and rosehip oil (0.1-5% w/w), the cellulosic preparation is ethyl cellulose, and the sunscreen agent is zinc oxide having a particle size of between about 5-100 nm.

Preferably, the preferred form further comprises about 0.5-1% w/w preservative, such as BHT.

Preferably, the preferred form further comprises about 0.5-5% w/w vitamin E acetate.

According to a sixth aspect of the present invention, there is provided a therapeutic composition for skin, said composition comprising resin, flexibilizer and at least one therapeutic agent, wherein upon application to skin, said composition forms a waterproof therapeutic flexible film.

According to a seventh aspect of the present invention, there is provided a method for prevent or treating a disorder of the skin, said method comprising the step of applying to the skin a composition comprising resin, flexibilizer and at least one therapeutic agent, wherein upon application to skin, said composition forms a waterproof therapeutic flexible film.

According to an eighth aspect of the present invention, there is provided the use of a composition in the preparation of a medicament for the prevention or treatment of a skin disorder, said composition comprising resin, flexibilizer and at least one therapeutic agent, wherein said composition forms a waterproof therapeutic flexible film when applied to skin.

Any suitable type of therapeutic agent may be used. The composition may include one or more therapeutic agents. A suitable therapeutic agent may be an antimicrobial such as a bactericide or fungicide. Another suitable therapeutic agent may promote wound healing or have anti-inflammatory properties. Yet another suitable therapeutic agent may be a steroid for treating eczema or dermatitis. Such therapeutic agents are well known in the art. Examples of suitable therapeutic agents include vitamins, such as vitamin E, and rosehip oil. Examples of suitable growth factors are described, for example, in the specification published as WO92/09301 to the American National Red Cross.

The therapeutic composition may include a pigment and/or dye for coating discoloured skin or skin blemishes and/or a sunscreen agent.

As mentioned above, each of the above compositions may be formulated for application to the skin, for example, as a paste, cream, gel, liquid or aerosol. The composition may be applied by way of a sponge, brush or spray. The colour, strength, flexibility, plasticity, stability, opacity, viscosity may be readily optimised by adjusting the quantity of each ingredient of the composition. The same coating composition or different coating compositions may be applied to the skin in two or more layers, so as to achieve an optimal effect.

The term “comprise” and variants thereof such as “comprising” and “comprised” are used herein to denote the inclusion of a stated integer or integers, unless in the context of usage an exclusive interpretation of a term is required.

Preferred embodiments of the present invention will now be described by way of example with reference to the accompanying figures.

### BRIEF DESCRIPTION OF THE DRAWINGS

FIG. 1 shows a patient having a port wine stain on her arm;

FIG. 2 shows the patient of FIG. 1 treated with a coloured film forming coating composition according to an embodiment of the present invention;

FIG. 3 is a plot of percentage transmittance versus wavelength for a coloured film forming coating/sunscreen composition, according to an embodiment of the present invention;

FIG. 4 is a plot of percentage transmittance versus wavelength for a sunscreen composition, according to an embodiment of the present invention; and

FIGS. 5 to 8 are plots of percentage transmittance versus wavelength for a coloured film forming coating/sunscreen composition, according to an embodiment of the present invention.

### DETAILED DESCRIPTION OF THE EMBODIMENTS

### Example 1

### Preparation of a Coloured Film Forming Coating Composition and Use Thereof for Coating a Skin Blemish

This example describes the preparation of a coloured coating composition and its use in coating a skin blemish such that the blemish has the appearance of normal skin surrounding the blemish. The composition is particularly useful for coating birthmarks, moles, basal cell carcinomas, pigment-related disorders, scars, burns, acne, veins, tattoos, eczema, dermatitis and bruising. The composition can be specifically colour tailored to the needs of an individual.

In order to formulate an appropriate coloured coating composition for coating a skin blemish, several readings of colour (hue, chroma and lightness) were taken from the skin blemish and normal skin surrounding the skin blemish using a spectrophotometer, the procedure for which is described in detail in the applicant's co-pending International Patent Application entitled “A Colour Compensating System”. Colour readings were taken from the darkest region of the skin blemish and averaged. Colour readings were also taken from normal skin surrounding the skin blemish and averaged.

A computer database was then interrogated. The computer database contained information relating to the colour properties of the blemish and the colour properties of the skin, as well as information relating to the appropriate colour properties of the coating composition to apply to the blemish such that the coated blemish exhibits the colour of the surrounding skin. The preferred coating compositions have a degree of translucency such that the colour of the coated skin blemish comprises the additive affects of the colour of the coating and the underlying colour of the blemish.

The computer database may be used to select the coating composition. Alternatively, the computer database may provide information as to the desirable colour properties of the coating composition required to obtain the appropriate compensatory colour properties and this information may then be used to provide instructions to the user as to an appropriate coating composition to be prepared to obtain the desired compensatory colour properties.

Once the desired coloured coating composition had been determined, that coating composition was made up from its basic ingredients.

### A. Preparation of Primary Pigment and Dye Dispersions

A primary dispersion was made for each pigment and dye to be used in the coloured composition.

Primary dispersions were prepared using one or more of the following pigments and dyes:
Natural or synthetic iron oxides, black, red, yellow, brown, blended in various ratios.
Rutile titanium dioxide (micronised).
Zinc oxide (micronised).
Ultramarine blue (micronised).
Mixed metal oxide (cobalt) blue, black, turquoise, green.
Bon arymadide red pigments.
Bon red (calcium).
Rubine toners.
Arylamide yellows.
Transparent iron oxide pigments.
Phthalocyanine blues.
Dioxazine violets.
Pearlescent pigments in various colours, including white, copper, bronze.
Solvent dyes red, orange, yellow, blue, violet, brown, black.

Such pigments and dyes (as well as other suitable pigments and dyes) are available from Redox Chemicals, Polyimpex, Avlo Australia, Clariant Australia, Shepherd International, Merk Australia, Ravenswood Australia, HCA Colours, Degussa Australia, Quantum Chemicals, Multichem, GCI Chemicals Australia, Orica Australia, Tradechem, Johnson and Mathey, Elementis, Bayer Chemicals Pty Ltd, BASF Australia, DIC International, APS Australia, and OMYA Southern.

Each pigment or dye 5-60% w/w was individually mixed to balance with castor oil (to 100% w/w) using a high-speed disperser for approximately 20 minutes, until the pigment or dye was fully wetted.

Each resulting liquid/paste was processed through a bead mill or triple roll mill until a dispersion was achieved of less than 5 micron particle size. Particle size and agglomeration parameters were checked, e.g. using a Hegman guage. Each primary pigment and dye dispersion was then stored in a respective container.

### B. Preparation of a Film Forming Composition

Shellac in both solid and liquid form is available from Redox Chemicals, Avlo Australia as well as from Quantum Chemicals. There are many forms of natural shellac available which vary greatly in colour from dark amber to blonde honey colour. A bleached version was used to give minimum coloration to the coloured composition.

De-waxed (blonde) shellac flakes 28% w/w were added slowly to ethanol 55% w/w and stirred until all of the shellac had dissolved. The solution was strained through a 10 micron filter cloth and then the following ingredients were added: ethyl cellulose 4% w/w; castor oil 4% w/w; inorganic thixotrope 4% w/w; preservatives 2% w/w and ethanol to 100% w/w.

A suitable preservative may include, for example, one or more of the following: butylated hydroxytoluene, butylated hydroxyanisole, hydroquinone and methylhydroquinone. Such preservatives may be obtained from Multichem, Redox Chemicals, APS Australia, Quantum Chemicals and Johnson Mathey.

The solution was stirred with gentle heating until all of the solids had dissolved and then strained through a 10 micron filter cloth. The solution was then stored in a container.

### C. Preparation of a Blend of Primary Pigment and Dye Dispersions

From the computer/spectrophotometer prediction, select primary pigment and dye dispersions (from part A.) were blended together using a high-speed mixer until homogenous—eg. white (“vehicle”) 86.16% w/w, black 03.77% w/w, red 0.58% w/w, yellow ochre 10.88% w/w. A white base (vehicle) was usually prepared from a dispersion of zinc oxide or titanium dioxide.

### D. Preparation of a Coloured Film Forming Coating Composition

A final coloured film forming coating composition was then prepared as follows:

| | |
|---|---|
| blend of pigment and dye dispersions (from part C.) | 12% w/w |
| film forming composition (from part B.) | 88% w/w |

The coloured coating composition was mixed vigorously and then applied to skin using brushing, sponging, or airbrushing.

The composition may be readily applied to large skin areas using an airbrush gun. For airbrushing, the final coloured film forming composition 30% volume by volume (v/v) may be diluted in ethanol 70% v/v. The composition is preferably applied to the skin in a cross-hatching manner.

For brushing or sponging, the coloured film forming composition may be used without dilution. A sponge having pores of an appropriate size can produce a stippled effect on the skin. The skin is usually cleaned with soapy water (to remove oils and chemical residues) prior to applying the composition.

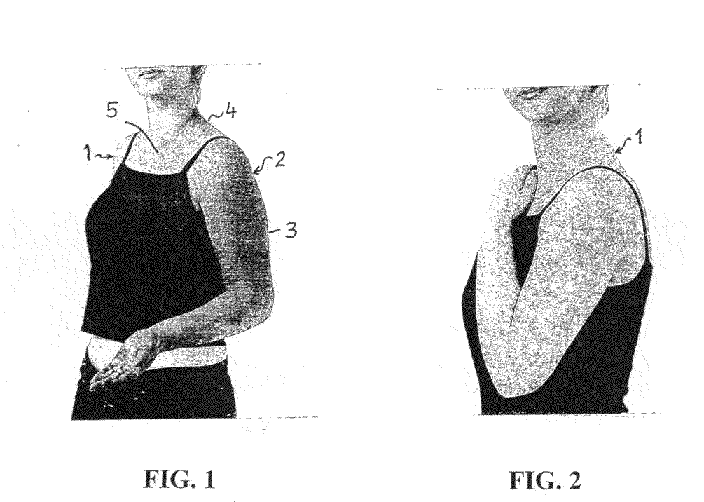

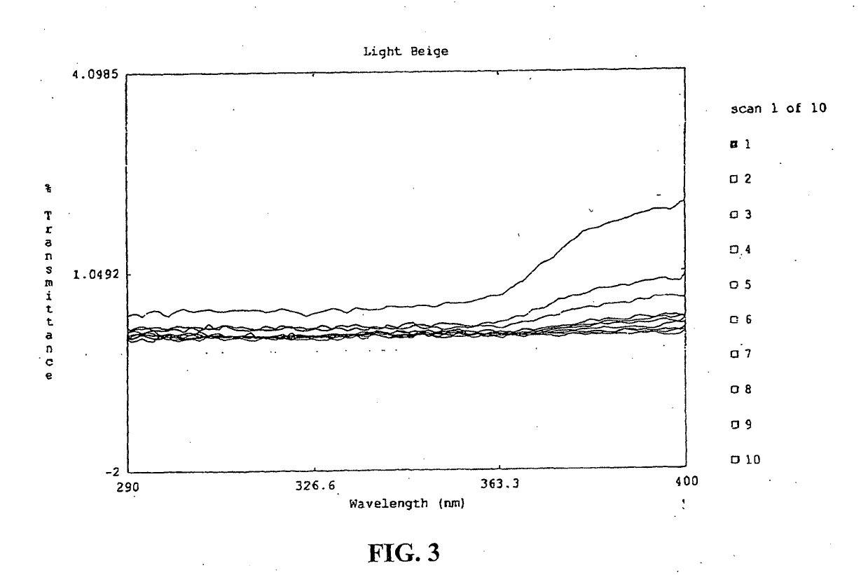

FIG. 1 shows a patient 1 having a port wine stain 2 extending over her arm 3, neck 4 and chest 5. FIG. 2 shows the same patient 1 but with the port wine stain 2 coated with a coloured composition prepared in accordance with Example 1.

### Example 2

### Preparation of a Coloured Film Forming Coating Composition and Use Thereof for Coating a Skin Blemish

This example describes the preparation of another preferred coloured coating composition and its use in coating a skin blemish such that the blemish has the appearance of normal skin surrounding the blemish.

### A. Preparation of Primary Pigment and Dye Dispersions

A primary dispersion was made for each pigment and dye to be used in the coloured composition. Primary dispersions were prepared as described in part A. of Example 1 except that the quantity of each pigment or dye varied and was balanced to 100% w/w using triple refined castor oil.

### B. Preparation of a Film Forming Composition

A film forming composition was prepared as described in part B. of Example 1 except that the composition comprised:

| | | |
|---|---|---|
| de-waxed (blonde) shellac flakes | 10.37% | w/w; |
| ethyl cellulose | 6.10% | w/w; |
| castor oil | 13.34% | w/w; |
| rosehip oil | 0.99% | w/w; |
| BHT | 1.23% | w/w; |
| fumed silica | 4.17% | w/w; and |
| ethanol | 63.80% | w/w. |

### C. Preparation of a Blend of Primary Pigment and Dye Dispersions

As described in part C. of Example 1, select primary pigment and dye dispersions (from part A.) were blended together.

### D. Preparation of a Coloured Film Forming Coating Composition

A final coloured film forming coating composition was then prepared as follows:

| | |
|---|---|
| blend of pigment and dye dispersions (from part C.) | 12% w/w |
| film forming composition (from part B.) | 88% w/w |

The coloured coating composition was mixed and then applied to skin using brushing, sponging, or airbrushing. For airbrushing, the final coloured film forming composition 30% volume by volume (v/v) may be diluted in ethanol 70% v/v.

Shellac, ethyl cellulose, castor oil, fumed silica and ethanol are the minimum ingredients required to form the film.

If a matte effect is desired, a fixing powder may be further applied to the coated skin. The fixing powder may be initially applied by sponge immediately after application of the composition and then reapplied after washing the skin. The fixing powder comprised:

| | | |
|---|---|---|
| 92.99% | w/w | talc; |
| 4.65% | w/w | zinc stearate; |
| 1.43% | w/w | silicone oil; and |
| 0.93% | w/w | Unigerm G2 ™ (a mixture of methylparaben, ethyl- |
| | | paraben, propylene glycol and mydazolidinyl urea). |

### Example 3

### Preparation of a Film Forming Sunscreen Composition and the Use Thereof

This example describes the preparation of a sunscreen composition and the use thereof. The sunscreen composition may protect individuals from premature ageing of skin, skin cancer and other harmful effects of solar radiation. Since the sunscreen film is long-lasting and has a high SPF value, it will be of particular use to those who are exposed to sunlight for long periods of time (e.g. sportsmen).

### A. Preparation of a Film Forming Composition

De-waxed (blonde) shellac flakes 28% w/w were added slowly to ethanol 55% w/w and stirred until all of the shellac had dissolved. The solution was strained through a 10 micron filter cloth and then the following ingredients were added: ethyl cellulose 4% w/w; castor oil 4% w/w; inorganic thixotrope 4% w/w; preservatives 2% w/w and ethanol to 100% w/w. The solution was stirred with gentle heating until all of the solids had dissolved and then strained through a 10 micron filter cloth. The solution was stored in a container.

### B. Preparation of a Nano Zinc Oxide Dispersion

Inorganic zinc oxide was milled to a nano scale, of less then 100 nm particle size. High energy milling in dry form was used to induce chemical reactions during ball-powder collisions to form nano particles in a solid-state matrix.

Agglomeration was minimized by ensuring that the particles were encapsulated on formation by a solid diluent phase (typically sodium chloride). The solid diluent phase was removed by a basic washing technique. This process formed equiaxed nano particles with a very narrow size distribution and very low levels of agglomeration.

The solid diluent phase allowed the particles to be heat treated without any agglomeration occurring. The heat treatment step ensured the product was completely reacted, removed all residual chemicals and stabilized the surfaces of the particles. The stabilized surfaces assisted in decreasing the reactivity of the particles, which aided in the subsequent dispersion in both aqueous and non-aqueous phases, and limited the generation of free radicals.

Zinc oxide becomes transparent when processed to nano particle size but still retains its UV protection properties. Due to its high refractive indices, zinc oxide blocks UV by both scattering and band gap absorption. A 2040 nm particle size was selected to provide broad spectrum UVB and UVA protection with maximum transparency in excess of 80%.

Dry milled 20-40 nm zinc oxide (40% w/w) was added slowly to the film forming composition of part A. (60% w/w) and mixed for a minimum of 20 minutes until a homogenous dispersion had been produced. The nano zinc oxide dispersion was then strained through a 10 micron filter cloth and stored in a container.

### C. Preparation of a Film Forming Sunscreen Composition

A final film-forming sunscreen composition was made up as follows and mixed vigorously before application to the skin:

| | |
|---|---|
| film forming composition (of part A.) | 70% w/w |
| nano zinc oxide dispersion (of part B.) | 30% w/w |

The sunscreen composition was then applied using brushing, sponging, or airbrushing.

### Example 4

### Sun Protection Factor Value of a Sunscreen Composition Containing Micronised Zinc Oxide

A coloured coating composition was prepared in accordance with Example 1 and had the colour blend: blue 0.60% w/w; red 0.53% w/w; yellow ochre 11.25% w/w; and white (vehicle) 47.60% w/w. Since the vehicle comprised micronised zinc oxide, the coloured coating composition also functioned as a sunscreen.

The composition was analysed using a Labsphere SPF Analyser with the following parameters:
substrate: MimSkin® on quartz
film thickness: 2 mg/cm²
number of scans: 10
UVBIUVA cut off: 320 nm

The results are summarised in the following tables:

Brush application - single film layer

| | | | | | | | Critical |
|---|---|---|---|---|---|---|---|
| | | UVA | Range | Range | Star | Cate- | Wave- |
| SPF | SD | Ratio | Lower | Higher | Rating | gory | length |
| 2.10 | 0.12 | 0.92 | off | off | **** | Maxi- | 388 nm |
| | | | scale | scale | | mum | |

Sponge application - single film layer

| | | | | | | | Critical |
|---|---|---|---|---|---|---|---|
| | | UVA | Range | Range | Star | Cate- | Wave- |
| SPF | SD | Ratio | Lower | Higher | Rating | gory | length |
| 1.56 | 0.03 | 0.86 | 0.80 | 0.92 | **** | Maxi- | 388 nm |
| | | | | | | mum | |

Application of four film layers/quartz

| | | | | | | | Critical |
|---|---|---|---|---|---|---|---|
| | | UVA | Range | Range | Star | Cate- | Wave- |
| SPF | SD | Ratio | Lower | Higher | Rating | gory | length |
| 1216.65 | 630.09 | 0.94 | off | off | **** | Maxi- | 387 nm |
| | | | scale | scale | | mum | |

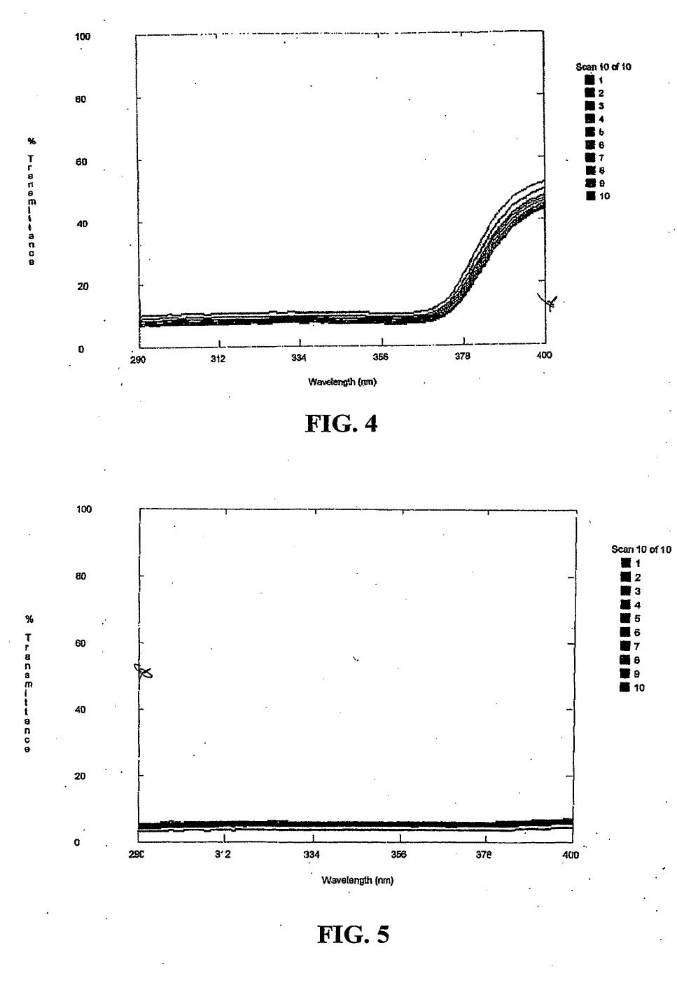

The final table shows that multilayering the film on skin (four layers) is likely to provide a much higher SPF value, in this case being 1216.65. FIG. 3 is a plot of percentage transmittance versus wavelength for the composition when applied as four layers, and indicates that the composition can provide broad UV protection.

### Example 5

### Preparation of a Film Forming Therapeutic Sunscreen Composition

This example describes the preparation of a preferred therapeutic sunscreen composition.

A film forming composition was prepared as described in parts A., B. and C. of Example 3 except that the final composition comprised:

| | | |
|---|---|---|
| de-waxed (blonde) shellac flakes | 12.96% | w/w; |
| ethyl cellulose | 7.79% | w/w; |
| castor oil | 1.14% | w/w; |
| rosehip oil | 0.50% | w/w; |
| BHT | 1.00% | w/w; |
| vitamin E acetate | 0.50% | w/w; |
| nano zinc oxide (Zinclear ™) | 34.40% | and |
| ethanol | 41.44% | w/w. |

Shellac, ethyl cellulose, castor oil, nano zinc oxide and ethanol are the minimum ingredients required to form the film.

The composition contains the therapeutic agent vitamin E acetate. This agent is an antioxidant and can decrease the effects of psoriasis, erythema and scaring from wounds. If desired, other therapeutic agents (eg. growth factors, steroids) may be used in the composition.

### Example 6

### Sun Protection Factor Value of a Sunscreen Composition Containing Nano Zinc Oxide

The therapeutic composition of Example 5 was analysed using a Labsphere SPF Analyser with the following parameters:

substrate: MimSkin® on quartz

rub-in method

film thickness: 2 mg/cm²

number of scans: 10

UVB/UVA cut off: 320 nm

The results are summarised in the following tables:

Pre photodegradation

| | | | | | | | Critical |
|---|---|---|---|---|---|---|---|
| | | UVA | Range | Range | Star | Cate- | Wave- |
| SPF | SD | Ratio | Lower | Higher | Rating | gory | length |
| 11.68 | 1.32 | 0.82 | 0.77 | 0.88 | ***** | Ultra | 377 nm |

Post photodegradation

| | | UVA | Range | Range | Star | | Photo- | Critical |
|---|---|---|---|---|---|---|---|---|
| SPF | SD | Ratio | Lower | Higher | Rating | Category | stability | Wavelength |
| 11.32 | 1.62 | 0.82 | 0.77 | 0.88 | ***** | Ultra | Yes | 377 nm |

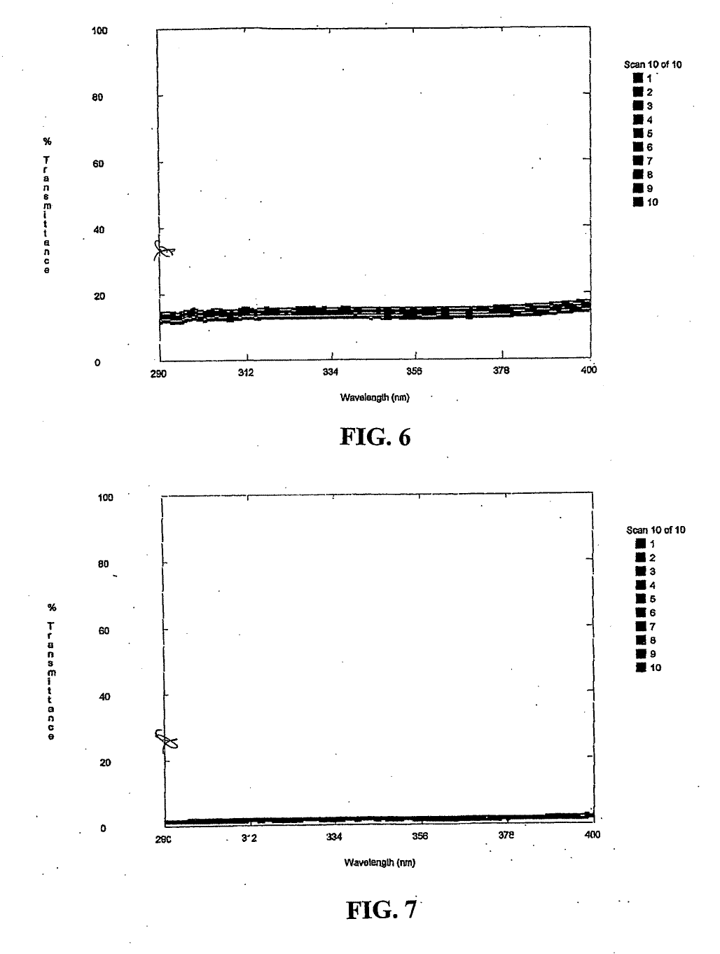

FIG. 4 is a plot of percentage transmittance versus wavelength for the composition when applied as a single layer. As for the composition tested in Example 4, multilayering the film on skin (four layers) provided a much higher (and more than additive) SPF value, and provided broad UV protection (results not shown).

### Example 7

### Sun Protection Factor Value of a Coloured Sunscreen Composition

A coloured coating composition was prepared in accordance with Example 2.

The following primary dispersions were prepared (to 100% w/w balance with castor oil):

| | |
|---|---|
| black iron oxide (Elementis) | 25% w/w |
| rubine bright red (Polyimpex) | 20% w/w |
| white (Tronox ® CR-828 titanium dioxide) | 50% w/w |
| yellow ochre | 40% w/w |

The following primary dispersion quantities were mixed with 44 g of the film forming composition described in part B. of Example 2: 0.03 g black iron oxide; 0.07 g rubine bright red; 4.13 g white; and 1.48 g yellow ochre.

Since the composition contained titanium dioxide, the coloured coating composition could also function as a sunscreen.

The composition, applied in 1 to 4 layers, was analysed using a Labsphere SPF Analyser with the following parameters:

substrate: MimSkin® on quartz

rub-in method

film thickness: 2 mg/cm²

number of scans: 10

UVB/UVA cut off: 320 nm

The results are summarised in the following tables:

Spray application - single film layer

| | | | | | | | Critical |
|---|---|---|---|---|---|---|---|
| | | UVA | Range | Range | Star | Cate- | Wave- |
| SPF | SD | Ratio | Lower | Higher | Rating | gory | length |
| 7.2 | 0.61 | 0.98 | Too | Too | ***** | Ultra | 389 nm |
| | | | high | high | | | |

Spray Application - two film layers

| | | UVA | Range | Range | Star | | Photo- | Critical |
|---|---|---|---|---|---|---|---|---|
| SPF | SD | Ratio | Lower | Higher | Rating | Category | stability | Wavelength |
| 19.6 | 3.06 | 0.98 | Too | Too | ***** | Ultra | Yes | 389 nm |
| | | | high | high | | | | |

Spray Application - three film layers

| | | UVA | Range | Range | Star | | Photo- | Critical |
|---|---|---|---|---|---|---|---|---|
| SPF | SD | Ratio | Lower | Higher | Rating | Category | stability | Wavelength |
| 53.42 | 11.98 | 0.98 | Too | Too | ***** | Ultra | Yes | 389 nm |
| | | | high | high | | | | |

Spray Application - four film layers

| | | UVA | Range | Range | Star | | Photo- | Critical |
|---|---|---|---|---|---|---|---|---|
| SPF | SD | Ratio | Lower | Higher | Rating | Category | stability | Wavelength |
| 115.21 | 21.74 | 0.98 | Too | Too | ***** | Ultra | Yes | 389 nm |
| | | | high | high | | | | |

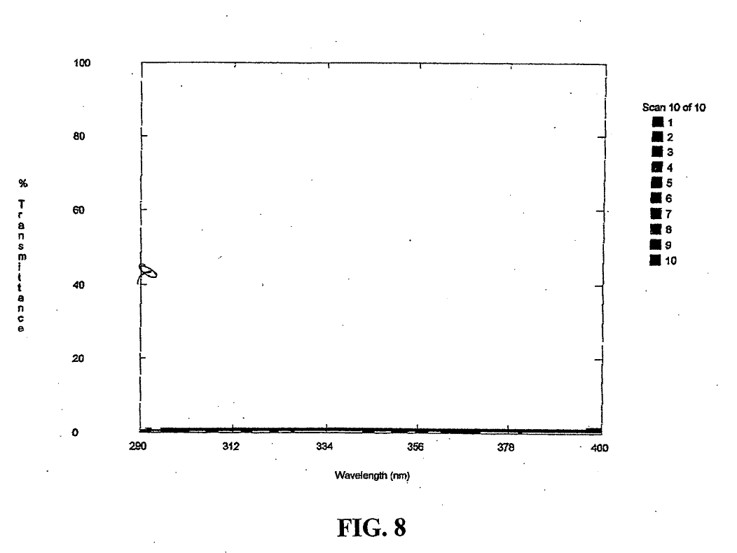

The final table shows that multilayering the film on skin (four layers) is likely to provide a much higher SPF value, in this case being 115.21. FIGS. 5-8 are plots of percentage transmittance versus wavelength for the composition when applied as one to four layers, respectively, and indicate that the composition can provide broad UV protection.

The coating compositions as exemplified herein have one or more of the following advantages: they are flexible and durable on the skin (typically lasting between 1 to 5 days);

they may be multilayered to provide a superior effect;

they are waterproof and sweat proof;

they remain intact even when immersed in hot water;

they bond to the skin when immersed in saltwater;

they remain intact even if clothing or a shaver is scraped over the film

they help keep the skin sterile;

they do not cause skin blemishes (pimples, breakouts);

they are gentle to the skin; and

they are easy to apply to the skin.

Whilst the above has been given by way of illustrative example of the invention, many modifications and variations may be made thereto by persons skilled in the art without departing from the broad scope and ambit of the invention as herein set forth.

1. A coating composition for skin, said composition comprising resin and flexibilizer, wherein upon application to skin, said composition forms a waterproof flexible film.

2. The coating composition of claim 1, wherein the flexible film is capable of remaining firmly adhered to the skin for about 2 to 3 days.

3. A coating composition for skin, said composition comprising:
about 5-30% w/w natural gum derivative;
about 50-80% w/w alcohol;
about 2-70% w/w oil;
about 2-10% w/w cellulosic preparation; and
about 0.5-10% w/w inorganic thickener,
wherein upon application to skin, said composition forms a waterproof flexible film.

4. The coating composition according to claim 3, wherein the natural gum derivative is shellac, the alcohol is ethanol, the oil is a mixture of 7-65% w/w castor oil and 0.1-5% w/w rosehip oil, the cellulosic preparation is ethyl cellulose, and the inorganic thickener is silicon dioxide.

5. A coloured coating composition for skin, said composition comprising resin, flexibilizer and pigment and/or dye, wherein upon application to skin, said composition forms a waterproof flexible coloured film.

6. A coloured coating composition for skin, said composition comprising:
about 5-30% w/w natural gum derivative;
about 50-80% w/w alcohol;
about 2-70% w/w oil;
about 2-10% w/w cellulosic preparation;
about 0.5-10% w/w inorganic thickener; and
at least one pigment and/or dye,
wherein upon application to skin, said composition forms a waterproof flexible coloured film.

7. A sunscreen composition for skin, said composition comprising resin, flexibilizer and sunscreen agent, wherein upon application to skin, said composition forms a waterproof flexible film having a defined sun protection factor (SPF) value.

8. A sunscreen composition for skin, said composition comprising:
about 5-30% w/w natural gum derivative;
about 20-60% w/w alcohol;
about 10-60% w/w sunscreen agent;
about 2-20% w/w oil; and
about 2-15% w/w cellulosic preparation,
wherein upon application to skin, said composition forms a waterproof flexible film having a defined sun protection factor (SPF) value.

9. The sunscreen composition of claim 8, wherein the sunscreen agent comprises zinc oxide.

10. The sunscreen composition of claim 8, wherein the natural gum derivative is shellac, the alcohol is ethanol, the oil is a mixture of 2-15% w/w castor oil and 0.1-5% w/w rosehip oil, the cellulosic preparation is ethyl cellulose, and the sunscreen agent is zinc oxide having a particle size of between about 5-100 nm.

11. The sunscreen composition of claim 7, wherein the flexible film is capable of remaining firmly adhered to the skin for about 2 to 3 days.

12. A therapeutic composition for skin, said composition comprising resin, flexibilizer and at least one therapeutic agent, wherein upon application to skin, said composition forms a waterproof flexible therapeutic film.

13. A therapeutic composition for skin, said composition comprising:
about 5-30% w/w natural gum derivative;
about 50-80% w/w alcohol;
about 7-70% w/w oil;
about 2-10% w/w cellulosic preparation;
about 0.5-10% w/w inorganic thickener; and
at least one therapeutic agent,
wherein upon application to skin, said composition forms a waterproof flexible therapeutic film.

14. The therapeutic composition of claim 13, wherein the therapeutic agent comprises about 0.5-5% w/w vitamin E acetate.

15. A method for coating a skin discoloration or skin blemish such that the skin discoloration or blemish blends with the surrounding skin, said method comprising the steps of:
(i) coating a skin discoloration or blemish with a composition comprising resin, flexibilizer, and pigment and/or dye; and
(ii) allowing the composition to form a waterproof flexible coloured film over the skin discoloration or blemish.

16. The method of claim 15, wherein said composition comprises:
about 5-30% w/w natural gum derivative;
about 50-80% w/w alcohol;
about 7-70% w/w oil;
about 2-10% w/w cellulosic preparation;
about 0.5-10% w/w inorganic thickener; and
at least one pigment and/or dye.

17. The method of claim 15, wherein the flexible coloured film has a degree of translucency when applied to the skin discoloration or blemish.

18. The method of claim 16, wherein the skin discoloration or blemish is selected from the group consisting of: a birthmark, a mole, a basal cell carcinoma, vitiligo, a scar, a burn, pigmentation, acne, a vein, tattoo, eczema, dermatitis and bruising.

19. The method of claim 16 further comprising the step of (iii) applying a fixing powder to the coated skin discoloration or blemish to create a matte effect and to produce a more natural skin appearance.

20. The method of claim 19, wherein the fixing powder comprises:
about 93.0% w/w talc;
about 4.7% w/w zinc stearate;
about 1.4% w/w silicone oil; and
about 0.9% w/w preservative.

21. The method of claim 16, wherein the composition is applied to the skin as a paste.

22. The method of claim 16, wherein 30% volume by volume (v/v) of the composition is diluted in 70% v/v ethanol and applied to the skin as a spray.

23. The method of claim 16, wherein said composition further comprises a sunscreen agent and said flexible film has a defined sun protection factor (SPF) value.

24. The method of claim 16, wherein said composition further comprises a therapeutic agent.

25. A method for prevent or treating a disorder of the skin, said method comprising the step of applying to the skin a composition comprising resin, flexibilizer and at least one therapeutic agent and/or sunscreen agent, wherein upon application to skin, said composition forms a waterproof flexible film containing said therapeutic agent and/or sunscreen agent.

26. The method of claim 25, wherein said disorder of the skin is xeroderma pigmentosum.
